(19) United States
(12) Patent Application Publication
(10) Pub. No.: US 2012/0294841 A1
(43) Pub. Date: Nov. 22, 2012

(54) PROBIOTIC MICROORGANISMS AS ACTIVE AGENTS AGAINST CHANGES IN THE SKIN'S MICRORELIEF

(75) Inventors: Audrey Gueniche, Rueil Malmaison (FR); Isabelle Castiel, Nice (FR)

(73) Assignees: NESTEC S.A., Vevey (CH); L'OREAL, Paris (FR)

(21) Appl. No.: 13/514,824

(22) PCT Filed: Dec. 7, 2010
(86) PCT No.: PCT/IB10/55630

§ 371 Date: Jul. 24, 2012

Related U.S. Application Data
(60) Provisional application No. 61/282,207, filed on Dec. 30, 2009.

(30) Foreign Application Priority Data
Dec. 8, 2009 (FR) 0958745

(51) Int. Cl. A61K 8/99; A61P 17/00; A61Q 19/00; A61Q 19/08; A61K 35/74; A61K 35/66

(52) U.S. Cl. 424/93.45; 424/93.1; 424/780; 424/93.51; 424/93.4

(57) ABSTRACT

The invention relates to the use of at least one probiotic microorganism as an active agent for preventing and/or treating impairments of the skin's microrelief.

The present invention relates to the use of probiotic microorganisms for smoothing the skin, especially facial skin.

More particularly, the invention relates to the use of a probiotic microorganism, especially of the genus Lactobacillus sp., as an active agent that is useful for treating and/or preventing microrelief impairment. The invention also relates to a process for smoothing the skin's microrelief.

The surface of human skin is not smooth. It has a relief reflected by fine lines, different from wrinkles, which may be seen with a magnifying glass in the case of children, and with the naked eye in the case of the elderly. These fine lines or grooves intercross so as to form structures of polygonal shapes, known as the skin's microrelief.

The number and depth of the grooves constituting the skin's microrelief may be affected by many external or internal factors. Among the extrinsic factors, mention may be made of exposure to sunlight, exposure to temperature and humidity variations, or exposure to pollutants. Among the intrinsic factors affecting the skin's microrelief, mention may be made of stress, tiredness, hormonal changes, dehydration of the epidermis or impairment of the skin's barrier function, or even ageing. These extrinsic and intrinsic factors have a tendency to increase the number and depth of the grooves of the microrelief.

These grooves become more pronounced and visible to the naked eye.

It thus proves to be important to be able to prevent or even reduce microrelief impairments.

Moreover, the external or internal factors that are liable to impair the skin's microrelief may also affect its complexion. Consequently, impairments of the microrelief and of the radiance of the complexion of the skin may often develop together.

WO 2007/112 996 describes a composition for smoothing the skin's microrelief, comprising various active agents extracted from plants, such as genistein, resveratrol or β-carotene, formulated in a fermented dairy product such as Nestlé LCI yoghurt.

WO 2006/104 730 describes a food supplement comprising probiotic microorganisms for improving the appearance of the skin, and preventing signs, especially age-related signs, on the skin, such as the appearance of wrinkles or a rough aspect.

U.S. 2008/206 211 describes the use of a culture medium conditioned with bacteria for treating dehydration or ageing of the skin.

However, it appears that none of the solutions proposed in the prior art proves to be satisfactory for preventing and/or treating impairments of the skin's microrelief.

Thus, there is still a need for novel active agents that are useful for treating and/or preventing impairment of the skin's microrelief, and in particular of facial skin.

There is also a need for novel active agents that are useful for reducing the number and/or depth of the grooves of the skin's microrelief.

There is still a need for novel active agents that are useful for improving the homogeneity of orientation of the fine lines of the microrelief and for increasing the density of corners/cm², which is characteristic of young skin.

There is also a need for novel active agents that can give the skin a luminous complexion.

There is also a need for novel active agents for preventing and/or treating skin imperfections.

The object of the present invention is to satisfy these needs.

According to a first subject, the present invention relates to a cosmetic use of at least one probiotic microorganism, especially of the genus Lactobacillus sp., as an active agent that is useful for treating and/or preventing impairment of the skin's microrelief.

The inventors have observed, surprisingly, that the administration of an effective amount, especially orally, of a probiotic microorganism, especially of the genus Lactobacillus sp., in particular Lactobacillus paracasei and more particularly Lactobacillus paracasei ST11, can significantly reduce the number and depth of the grooves of the microrelief, and increase the density of corners/cm².

According to one embodiment, the impairments of the skin's microrelief under consideration in the present invention are not age-related, and in particular are not signs of ageing of the skin.

The use of probiotic microorganisms according to the invention advantageously makes it possible to reinforce the skin's barrier function properties, thus promoting the integrity of the skin and preserving its microrelief.

According to another advantage, a use according to the invention makes it possible to optimize the assimilation of the nutrients supplied by food in the intestinal mucosae and to promote the supply of nutrients that are essential to cell metabolism. Synthesis of the various functional and structural components of the skin is thus promoted, allowing maintenance of the skin's homeostasis and reducing the impairments of the skin's microrelief, or even smoothing the skin.

According to yet another advantage, a use in accordance with the invention makes it possible to restore and to maintain the moisturization and the barrier function of the skin, and also of the subjacent tissues, thus promoting the reduction of the depth of the grooves of the skin's microrelief and reducing its visibility.

According to one embodiment, a probiotic microorganism under consideration in the invention is different from a Lactobacillus johnsonii formulated in a composition comprising, on a weight basis:
875.42 g of 1.7% whole milk,
10 g of 1.0% skimmed milk,
30 g of Nestlé LCI yoghurt,
70 g of sugar,
3 g of stabilizer,
0.08 g of tangerine flavouring,
0.93 g of Teavigo,
0.17 g of genistein TG,
0.14 g of resveratrol,
0.24 g of 7% β-carotene CWS,
10 g of ROPUFA “30” n-3 food oil.

According to one preferred embodiment, a probiotic microorganism that is suitable for use in the invention for treating and/or preventing impairment of the skin's microrelief may advantageously be a Lactobacillus paracasei and preferably a Lactobacillus paracasei ST11.

According to another preferred embodiment, a probiotic microorganism that is suitable for use in the invention is not combined with at least one active agent chosen from whole milk, skimmed milk, Teavigo, genistein, resveratrol, β-carotene and ROPUFA “30” n-3 food oil, and mixtures thereof.

According to another embodiment, a probiotic microorganism that is suitable for use in the invention is not combined with a mixture comprising whole milk, skimmed milk, Teavigo, genistein, resveratrol, β-carotene and ROPUFA “30” n-3 food oil.

According to yet another embodiment, a probiotic microorganism that is suitable for use in the invention is different from a Lactobacillus johnsonii (or acidophilus).

According to one embodiment, a subject of the present invention is also a cosmetic process for preventing and/or treating impairment of the skin's microrelief, comprising the administration of at least one probiotic microorganism, especially of the genus Lactobacillus sp., especially as defined hereinbelow.

According to the invention, the term “skin” is intended to denote all of the epidermis of an individual, in particular of a human being, and more particularly the skin of the neckline, the neck and the face, especially facial skin.

According to another embodiment, an object of the present invention is to reduce the number of grooves of the skin's microrelief and/or to reduce the depth of the grooves of the skin's microrelief.

According to another embodiment, an object of the present invention is to increase the density of corners/cm² of the skin.

A groove, or fine line, of the skin's microrelief has a maximum depth of 55 μm. A skin groove with a depth of greater than 55 μm may be termed a line (55 to 110 μm) or a wrinkle (greater than 110 μm).

Thus, the structures of the skin's microrelief under consideration in the invention have a depth not exceeding 55 μm.

The grooves or fine lines intercross and form structures of polygonal shapes, characteristic of the skin's microrelief, and are different from the wrinkles or lines of the skin. These structures define corners, whose presence in high number is characteristic of healthy, smooth and especially young skin.

The presence and visibility of the grooves or fine lines of the skin's microrelief that are under consideration in the present invention may be accentuated by various extrinsic or intrinsic factors such as exposure to temperature or moisture variations, exposure to sunlight or UV, exposure to cold, exposure to pollutants, exposure to stress or tiredness, or exposure to hormonal changes.

The presence or visibility of the grooves of the skin's microrelief may also be affected by the degree of moisturization of the skin or of the subjacent tissues.

A sparingly marked microrelief is a sign of good health of the skin, and especially of good moisturization and well-regulated homeostasis of the barrier function properties.

The use of a probiotic microorganism in accordance with the invention, especially of the genus Lactobacillus sp., advantageously makes it possible to prevent, treat or reverse the deleterious effects of these extrinsic or intrinsic factors on the skin's microrelief.

According to one embodiment, the microrelief under consideration in the present invention is advantageously the microrelief of facial skin.

According to one embodiment, a probiotic microorganism that is suitable for use in the invention may be administered orally, topically or parenterally, and in particular orally.

A use of a microorganism according to the invention necessarily takes place in an effective amount, i.e. an amount that allows the probiotic microorganism to display its active properties with regard to the impairments of the skin's microrelief that are to be prevented and/or treated.

For the purposes of the present invention, the term “prevent” means at least partly reducing the risk of manifestation of a given phenomenon, i.e., in the present invention, impairment of the skin's microrelief. A partial reduction implies that the risk remains, but to a lesser extent than before implementing the invention.

Microorganisms

For the purposes of the present invention, the term “probiotic microorganism” means a live microorganism, which, when consumed in adequate amount, has a positive effect on the health of its host (“Joint FAO/WHO Expert Consultation on Evaluation of Health and Nutritional Properties of Probiotic in Food Including Powder Milk with Live Lactic Acid Bacteria, 6 October 2001”), and which may in particular improve the intestinal microbial balance.

According to one embodiment, a probiotic microorganism according to the invention may be used in an isolated or purified form, i.e. not mixed with one or more compound(s) liable to be associated with it in its medium of origin.

According to another embodiment, a probiotic microorganism according to the invention may be used in a live, semi-active, inactivated or dead form.

According to one advantageous embodiment of the invention, a probiotic microorganism may advantageously be used in inactivated or dead form.

A probiotic microorganism administered topically may advantageously be used in inactivated or dead form.

A probiotic microorganism administered orally may advantageously be used in live form.

For the purposes of the invention, an “inactivated” microorganism is a microorganism that is no longer capable, temporarily or definitively, of forming colonies in culture. For the purposes of the invention, a “dead” microorganism is a microorganism that is no longer capable, definitively, of forming colonies in culture. Dead or inactivated microorganisms may have intact or ruptured cell membranes. Thus, the term “inactivated” also denotes microorganism extracts and lysates as detailed hereinbelow. Dead or inactivated microorganisms may be produced via any method known to those skilled in the art.

According to one advantageous embodiment, the probiotic microorganisms used according to the invention are at least partly inactivated or dead.

The expression “probiotic microorganisms that are at least partly inactivated or dead” denotes a preparation of probiotic microorganisms in accordance with the invention comprising at least 80%, in particular at least 85%, more particularly at least 90%, or even at least 95%, or at least 99% of inactivated or dead probiotic microorganisms relative to the total amount of non-inactivated live probiotic microorganisms contained in the initial preparation before being subjected to a process to inactivate or kill the microorganisms.

The degree of inactivation or of death obtained depends on the application conditions of the method used, which are adjusted by a person skilled in the art according to the degree of inactivation or death to be obtained.

According to one embodiment, the invention comprises the use of a preparation comprising 100% of inactivated or dead probiotic microorganisms.

An inactivated probiotic microorganism that is suitable for use in the invention may be prepared by irradiation, heat inactivation or lyophilization of a microorganism preparation. These methods are known to those skilled in the art.

More particularly, the inactivation of probiotic microorganisms by irradiation may comprise the use of gamma rays, x-rays, exposure to UV or heat, or a pressure reduction. The type of treatment, the intensity, the dose and the exposure time are adjusted by a person skilled in the art according to the amount and nature of the probiotic microorganisms to be inactivated.

According to one preferred embodiment variant, a probiotic microorganism that is suitable for use in the invention is used in an inactivated form obtained by irradiation, especially gamma irradiation.

Inactivation by lyophilization may be performed via any method known in the field. Advantageously, probiotic microorganisms inactivated by lyophilization may be recultured.

Heat inactivation may be performed by incubating the probiotic microorganisms of the invention for a prolonged period of time, for example at least two hours, at 170° C. Heat inactivation may also be performed by autoclaving, by subjecting the probiotic microorganisms of the invention to a temperature of 121° C. for a period of at least 20 minutes and at an atmospheric pressure of 2 bar.

Alternatively, the heat inactivation may be performed by subjecting the probiotic microorganisms to a freezing temperature, for a prolonged period of time.

A probiotic microorganism according to the invention may be used in whole form, i.e. essentially in its native form, or in the form of extracts or lysates of disintegrated suspensions comprising fractions and/or metabolites of this microorganism.

For the purposes of the invention, the term “metabolite” denotes any substance derived from the metabolism of the microorganisms, and especially secreted by the microorganisms under consideration according to the invention and also endowed with efficacy for the treatment and/or prevention of impairments of the skin's microrelief.

For the purposes of the invention, the term “fraction” more particularly denotes a fragment of the said microorganism that is endowed with efficacy for the treatment and/or prevention of impairments of the skin's microrelief, by analogy with the said whole microorganism.

An extract or lysate that is suitable for use in the invention may be prepared from the probiotic microorganisms at the end of the growth phase.

According to one preferred embodiment, a probiotic microorganism that is suitable for use in the invention may be used in the form of a lysate.

For the purposes of the invention, a “lysate” commonly denotes a material obtained after the destruction or dissolution of biological cells via a phenomenon known as cell lysis, thus giving rise to the release of the intracellular biological constituents naturally contained in the cells of the microorganism under consideration.

For the purposes of the present invention, the term “lysate” is used without preference to denote the whole lysate obtained via lysis of the microorganism under consideration or only a fraction thereof.

The lysate used is thus totally or partially formed from the intracellular biological constituents and from the constituents of the cell walls and membranes.

Advantageously, a lysate used for the invention may be the whole lysate obtained via lysis of the microorganism under consideration.

This cell lysis may be accomplished via various techniques, such as an osmotic shock, a heat shock, via ultrasonication, or alternatively under a mechanical stress of centrifugation type.

More particularly, this cell lysate may be obtained according to the technique described in patent U.S. Pat. No. 4,464,362, and especially according to the following protocol.

In particular, a lysate of the invention may be obtained via ultrasonic disintegration of a medium comprising probiotic microorganisms in suspension in order to release therefrom the cytoplasmic fractions, the cell wall fragments and the products derived from metabolism. All the components in their natural distribution are then stabilized in a weakly acidic aqueous solution.

A microorganism, an extract or a lysate may be used in various forms, such as a solution, a culture supernatant, a powder, optionally lyophilized, or a concentrate.

According to one embodiment, a probiotic microorganism that is suitable for use in the invention may be chosen from Lactobacillus sp., Bifidobacterium sp., Cocci, yeasts and sporulating bacteria, and mixtures thereof.

According to one embodiment, a microorganism that is suitable for use in the invention is preferentially chosen from:
ascomycetes: especially Saccharomyces, especially S. cerevisiae or S. boulardii, Yarrowia, especially Y. Lipolytica, Kluyveromyces, Torulaspora, Schizosaccharomyces pombe, Debaromyces, Candida, Pichia, Aspergillus, especially K. lactis and Penicillium,
sporulating bacteria: especially Bacillus sp., especially B. cereus var toyo or B. subtilis, B. coagulans, B. licheniformis, Escherichia sp., especially E. coli strain nissle, Propionibacterium sp., especially P. freudenreichii, Bacteroides sp., Fusobacterium sp., and Weissella sp.,
Cocci: especially Melissococcus sp., Staphylococcus sp., especially S. carnosus or S. xylosus, Peptostrepococcus sp., Pediococcus sp., especially P. acidilactici, Micrococcus sp., Leuconostoc sp., especially L. mesenteroides subsp dextranicum, Aerococcus sp., Oenococcus sp., Enterococcus sp., especially E. faecalis or E. faecium, Lactococcus sp., especially L. lactis subsp lactis or L. cremoris, Sporolactobacillus sp., especially S. inulinus, and Streptococcus sp., especially S. salivarius subsp. thermophilus or S. thermophilus,

Lactobacillus species: especially L. paracasei, L. acidophilus, L. amylovorus, L. casei, L. rhamnosus, L. brevis, L. crispatus, L. delbrueckii subsp. lactis, L. bulgaricus, L. fermentum, L. helveticus, L. gallinarum, L. gasseri, L. johnsonii, L. plantarum, L. reuteri, L. salivarius, L. alimentarius, L. curvatus, L. casei subsp. casei, L. sake, and
bifidobacteria or Bifidobacterium species: B. adolescentis, B. animalis, B. bifidum, B. breve, B. lactis, B. longum, B. infantis, B. pseudocatenulatum,
and mixtures thereof.

More particularly it may be a probiotic microorganism chosen from Lactobacillus sp., Sporolactobacillus sp., Enterococcus sp., Lactococcus sp., Bacillus sp., Streptococcus sp., Pediococcus sp., Leuconostoc sp. or Bifidobacterium sp., and in particular chosen from Lactobacillus sp., Bifidobacterium sp., and mixtures thereof.

As examples of probiotic microorganisms that are suitable for use in the invention, mention may be made of Bifidobacterium adolescentis, B. animalis, B. bifidum, B. breve, B. lactis, B. longum, B. infantis, B. pseudocatenulatum, Lactobacillus acidophilus NCFB 1748, L. paracasei, L. amylovorus, L. casei (Shirota), L. rhamnosus strain GG, L. brevis, L. crispatus, L. bulgaricus, L. delbrueckii subsp. lactis, L. fermentum, L. helveticus, L. gallinarum, L. gasseri, L. johnsonii CNCM 1-1225, L. plantarum, L. reuteri, L. salivarius, L. alimentarius, L. curvatus, L. casei subsp. casei, L. sake, Enterococcus faecalis, E. faecium, Lactococcus lactis, L. lactis subsp lactis, L. lactis subsp cremoris, Leuconostoc mesenteroides subsp. dextranicum, Pediococcus acidilactici, Sporolactobacillus inulinus, Streptococcus salvarius subsp. thermophilus, S. thermophilus,

Staphylococccus carnosus, S. xylosus, Saccharomyces cerevisiae, S. boulardii, Bacillus cereus var toyo, B. cereus var subtilis, B. coagulans, B. licheniformis, Escherichia coli strain nissle, Propionibacterium freudenreichii, and mixtures thereof.

As more specific examples of probiotic microorganisms that are suitable for use in the invention, mention may be made advantageously of Bifidobacterium bifidum, B. infantis, B. longum, Lactobacillus acidophilus, L. alimentarius, L. casei subsp. casei, L. casei shirota, L. paracasei, L. curvatus, L. delbrueckii subsp. lactis, L. gasseri, L. johnsonii, L. reuteri, L. rhamnosus (Lactobacillus GG), L. sake, Lactococcus lactis, Streptococcus thermophilus, Staphylococccus carnosus, S. xylosus, and mixtures thereof.

More particularly as probiotic microorganisms that are suitable for use in the invention, mention may be made of Lactobacillus johnsonii or acidophilus, L. reuteri, L. rhamnosus, L. paracasei, L. casei, L. alimentarius, L. curvatus, L. delbrueckii subsp. lactis, L. gasseri, L. sake, Bifidobacterium bifidum, B. breve, B. longum, B. animalis, B. lactis, B. infantis, B. adolescentis, B. pseudocatenulatum, and mixtures thereof.

The species that are most particularly suitable for use in the invention are Lactobacillus johnsonii, L. paracasei, Bifidobacterium adolescentis, B. longum deposited, respectively, according to the Treaty of Budapest, at the Institut Pasteur (28 rue du Docteur Roux, F-75024 Paris cedex 15) on 30 Jun. 1992, 12 Jan. 1999, 15 Apr. 1999 and 15 Apr. 1999 under the following designations CNCM I-1225, CNCM I-2116, CNCM I-2168 and CNCM I-2170, and the genus Bifidobacterium lactis (Bb 12) (ATCC27536) or Bifidobacterium longum (BB536). The strain of Bifidobacterium lactis (ATCC27536) may be obtained from Hansen (Chr. Hansen A/S, 10-12 Boege Alle, P.O. Box 407, DK-2970 Hoersholm, Denmark).

Advantageously, a microorganism that is suitable for use in the invention may be a lactic acid probiotic microorganism, which produces lactic acid by fermentation of sugar.

According to one preferred embodiment, a probiotic microorganism that is suitable for use in the invention, for example topically or orally, and especially orally, may in particular be a microorganism of the genus Lactobacillus sp.

Preferably, a microorganism of the genus Lactobacillus sp. that is suitable for use in the invention may be chosen from Lactobacillus johnsonii, L. acidophilus, L. reuteri, L. paracasei and L. casei, and mixtures thereof.

According to one preferred embodiment, a microorganism that is suitable for use in the invention may be chosen from Lactobacillus paracasei, L. johnsonii and L. acidophilus and mixtures thereof.

According to one preferred embodiment, a microorganism that is suitable for use in the invention may be a Lactobacillus paracasei.

According to another preferred embodiment, a microorganism that is suitable for use in the invention may in particular be the strain Lactobacillus paracasei ST11 deposited according to the Treaty of Budapest at the Institut Pasteur (28 rue du Docteur Roux, F-75024 Paris cedex 15) on 30 Jun. 1992 under the designation CNCM I-2116.

According to one embodiment, a probiotic microorganism that is suitable for use in the invention is used orally, parenterally or topically.

The term “parenteral” route means a route other than the oral and topical routes. A parenteral route that is suitable for use in the invention may be, for example, the nasal route.

According to one preferred embodiment, a probiotic microorganism that is suitable for use in the invention is used topically.

The topical route advantageously makes it possible to obtain local action with regard to the desired effect.

According to another preferred embodiment, a probiotic microorganism that is suitable for use in the invention is used orally or parenterally, and in particular orally.

The oral route or the parenteral route advantageously makes it possible to obtain a global action with regard to the desired effect.

A microorganism of the invention may be formulated in a composition in a proportion of at least 0.0001% expressed as dry weight, in particular in a proportion from 0.0001% to 30%, in particular in a proportion from 0.001% to 20% and more particularly in a proportion from 0.01% to 15% by weight, in particular from 0.1% to 10% by weight and especially from 1% to 5% by weight relative to the total weight of the composition containing it.

In general, a composition according to the invention intended to be administered orally may comprise for the live microorganisms from 10³ to 10¹⁵ cfu/g, in particular from 10⁵ to 10¹⁵ cfu/g and more particularly from 10⁷ to 10¹² cfu/g of live microorganisms per gram of composition, or equivalent doses calculated for inactivated or dead microorganisms or for microorganism fractions or for produced metabolites.

In particular, in a composition administered orally, the corresponding microorganism and/or fraction and/or metabolite concentration may be adjusted so as to correspond to doses, expressed as microorganism equivalent, ranging from 5×10⁵ to 10¹³ cfu/day and in particular from 10⁸ to 10¹¹ cfu/day.

A composition for topical application according to the invention may generally comprise from 0.0001% to 30% by weight of microorganisms per gram of composition, in particular a proportion of from 0.001% to 20%, more particularly a proportion of from 0.01% to 15% by weight, in particular from 0.1% to 10% by weight and especially from 1% to 5% by weight of microorganisms relative to the total weight of the composition containing it.

In the particular case of topical administration, it may be advantageous to use microorganisms in inactivated or even dead form, especially in the form of an extract or a lysate.

A microorganism may also be included in a composition in the form of fractions of cell components or in the form of metabolites, in particular in the form of a lysate. The microorganism(s), metabolite(s) or fraction(s) may also be introduced in the form of a lyophilized powder, a culture supernatant and/or, where appropriate, in a concentrated form.

When a composition comprises metabolites, the contents of metabolites in the compositions correspond substantially to the contents that may be produced by 10³ to 10¹⁵ cfu, in particular 10⁵ to 10¹⁵ cfu and more particularly 10⁷ to 10¹² cfu of live microorganisms per gram of composition.

Expression of the amount of metabolites or fractions of a microorganism in “cfu”, or of dead microorganisms, is intended to denote the amount of this microorganism used for the preparation of the metabolites or fractions of this microorganism.

Compositions

A probiotic microorganism that is suitable for use in the invention is advantageously formulated in a composition that may be in any galenical form normally available for the selected mode of administration.

A composition according to the invention comprises a physiologically or pharmaceutically acceptable medium.

A composition according to the invention may be administered orally, parenterally, especially subcutaneously or intradermally, or topically.

Preferably, a composition of the invention may be administered topically.

According to one embodiment, a topical composition according to the invention may advantageously be formulated in any galenical form that is suitable for caring for the skin and mucous membranes and may be in the form of ointments, creams, milks, pomades, powders, impregnated pads, solutions, gels, sprays, lotions or suspensions. They may also be in the form of microspheres or nanospheres or lipid or polymer vesicles or polymer patches and hydrogels allowing controlled release. These topical compositions may be either in anhydrous form or in aqueous form according to the dermocosmetic indication.

A composition intended for topical administration may be an aqueous, aqueous-alcoholic or oily solution, a solution or dispersion of the lotion or serum type, an emulsion of liquid or semi-liquid consistency of the milk type, obtained by dispersing a fatty phase in an aqueous phase (O/W) or conversely (W/O), a suspension or an emulsion of soft, semi-solid or solid consistency, of the cream type or of the aqueous or anhydrous gel type, a multiple emulsion (W/O/W or O/W/O), a microemulsion, a nanoemulsion, a preparation of microcapsules, a preparation of microparticles, a vesicular dispersion of ionic and/or nonionic type, or a wax/aqueous-phase dispersion.

According to one preferred embodiment, a topical composition may be in the form of a solution, a cream, a gel, an emulsion, a mousse or an aerosol composition containing a propellant.

According to one preferred embodiment, a topical composition may also be in the form of a transdermal system for active or passive release of the active agent(s) transdermally, for example of patch or gel patch (hydrogel) type.

These compositions are prepared according to the usual methods.

A composition according to the invention may constitute a composition for treating or caring for the skin or the scalp, or an antisun or artificial-tanning composition, or alternatively a skin-cleansing or makeup-removing product, a deodorant product or a fragrancing compound.

Such a composition may then be uncoloured or weakly coloured, and may optionally contain additional cosmetic or dermatological active agents, especially as indicated hereinbelow. It may then be used as a care base for the skin or the lips, for example in the form of a lip balm, protecting the lips against the cold and/or sunlight and/or the wind, or as a day or night care cream for facial and/or bodily skin.

It may also be in the form of a medicated or unmedicated, colouring or non-colouring shampoo, or a hair conditioner.

A composition according to the invention may also constitute a coloured cosmetic composition and especially a makeup composition for the skin and/or mucous membranes. In particular, such a composition may be a foundation, a blusher, a face powder, an eyeshadow, a concealer compound in stick form, a lipstick or a lip gloss, optionally with care or treating properties. Preferably, it may be a coloured (beige or green) makeup composition for correcting the colour of the complexion.

A composition administered topically may especially constitute a cleansing, protective, treating or care cream, a skincare lotion, gel or mousse, a cleansing or disinfecting lotion, a bath composition or a deodorant composition.

A composition according to the invention may also consist of a solid preparation constituting a cleansing soap or bar.

When a composition of the invention is an emulsion, the proportion of the fatty phase may range from 5% to 80% by weight and preferably from 10% to 50% by weight relative to the total weight of the composition. The oils, emulsifiers and co-emulsifiers used in the composition in emulsion form are chosen from those conventionally used in cosmetics and/or dermatology. The emulsifier and the co-emulsifier may be present in the composition in a proportion ranging from 0.3% to 30% by weight and preferably from 0.5% to 20% by weight relative to the total weight of the composition.

When the composition of the invention is an oily gel or solution, the fatty phase may represent more than 90% of the total weight of the composition.

In a known manner, galenical forms intended for topical administration may also contain adjuvants that are common in the cosmetic, pharmaceutical and/or dermatological field, such as hydrophilic or lipophilic gelling agents, hydrophilic or lipophilic active agents, preserving agents, antioxidants, solvents, fragrances, fillers, screening agents, odour absorbers and dyestuffs. The amounts of these various adjuvants are those conventionally used in the field under consideration, for example from 0.01% to 20% of the total weight of the composition. Depending on their nature, these adjuvants may be introduced into a fatty phase and/or into the aqueous phase.

As fatty substances that may be used in the invention, mention may be made of mineral oils, for instance hydrogenated polyisobutene and liquid petroleum jelly, plant oils, for instance the liquid fraction of shea butter, sunflower oil and apricot kernel oil, animal oils, for instance perhydrosqualene, synthetic oils, especially purcellin oil, isopropyl myristate and ethylhexyl palmitate, unsaturated fatty acids, and fluoro oils, for instance perfluoropolyethers. Fatty alcohols, fatty acids, for instance stearic acid, and, for example, waxes, especially paraffin wax, carnauba wax and beeswax, may also be used. Silicone compounds may also be used, for instance silicone oils, for example cyclomethicone and dimethicone, and silicone waxes, resins and gums.

As emulsifiers that may be used in the invention, examples that may be mentioned include glyceryl stearate, polysorbate 60, the mixture of cetylstearyl alcohol/cetylstearyl alcohol oxyethylenated with 33 mol of ethylene oxide, sold under the name Sinnowax AO® by the company Henkel, the mixture of PEG-6/PEG-32/glycol stearate sold under the name Tefose® 63 by the company Gattefossé, PPG-3 myristyl ether, silicone emulsifiers such as cetyldimethicone copolyol, and sorbitan monostearate or tristearate, PEG-40 stearate and oxyethylenated (20 OE) sorbitan monostearate.

As solvents that may be used in the invention, mention may be made of lower alcohols, especially ethanol and isopropanol, and propylene glycol.

The composition of the invention may also advantageously contain a spring and/or mineral water, chosen especially from Vittel water, waters from the Vichy basin, and Roche Posay water.

Hydrophilic gelling agents that may be mentioned include carboxylic polymers such as carbomer, acrylic copolymers such as acrylate/alkylacrylate copolymers, polyacrylamides and especially the mixture of polyacrylamide, C₁₃₋₁₄ isoparaffin and Laureth-7 sold under the name Sepigel 305® by the company SEPPIC, polysaccharides, for instance cellulose-based derivatives such as hydroxyalkylcelluloses and in particular hydroxypropylcellulose and hydroxyethylcellulose, natural gums such as guar gum, locust bean gum and xanthan gum, and clays.

Lipophilic gelling agents that may be mentioned include modified clays, for instance bentones, metal salts of fatty acids, for instance aluminium stearates, and hydrophobic silica, or alternatively ethylcellulose and polyethylene.

In the case of a composition in accordance with the invention for oral administration, the use of an ingestible support is preferred.

The ingestible support may be of diverse nature according to the type of composition under consideration.

Tablets, gel capsules or lozenges, suspensions, oral supplements in dry form and oral supplements in liquid form are thus especially suitable for use as food supports.

Milk, yoghurt, cheese, fermented milks, milk-based fermented products, ice creams, cereal-based products or fermented cereal-based products, milk-based powders, baby and infant formulas, food products of confectionery type, chocolate, cereals or animal feed, in particular for pets, are also suitable for use as food supports.

The term “oral composition” means, for example, nutritional, nutraceutical, cosmeceutical or pharmaceutical compositions, comprising at least one probiotic microorganism according to the invention.

Formulation of the oral compositions according to the invention may be performed via any usual process known to those skilled in the art for producing drinkable solutions, sugar-coated tablets, gel capsules, gels, emulsions, tablets to be swallowed or chewed, wafer capsules, especially soft or hard wafer capsules, granules to be dissolved, syrups, solid or liquid foods, and hydrogels allowing controlled release.

In particular, a probiotic microorganism according to the invention may be incorporated into any form of food supplement or enriched food, for example food bars, or compacted or loose powders. The powders may be diluted with water, with soda, with dairy products or soybean derivatives, or may be incorporated into food bars.

According to one embodiment, a composition according to the invention administered orally may be formulated in the form of sugar-coated tablets, gel capsules, gels, emulsions, tablets, wafer capsules, hydrogels, food bars, compacted or loose powders, liquid suspensions or solutions, confectioneries, fermented milks, fermented cheeses, chewing gum, toothpaste or spray solutions.

The oral compositions may be either in anhydrous form or in aqueous form according to the dermocosmetic indication.

A probiotic microorganism of the invention may moreover be formulated with the excipients and components that are common for such oral compositions or food supplements, i.e. especially fatty and/or aqueous components, humectants, thickeners, preserving agents, texturizers, flavour enhancers and/or coating agents, antioxidants and preserving agents.

Formulating agents and excipients for oral compositions, and especially for food supplements, are known in this field and will not be the subject of a detailed description herein.

According to the preferred indication of the use of a probiotic microorganism of the invention, a composition of the invention will be a cosmetic, dermatological or pharmaceutical composition.

In particular, the composition according to the invention may be a food composition for human consumption. It may in particular be a case of nutritional whole foods, drinks, mineral waters, soups, dietary supplements and replacement foods, nutritional bars, confectioneries, fermented or unfermented milk-based products, yoghurt products, milk-based powders, enteral nutritional products, baby and/or infant compositions, fermented or unfermented cereal-based products, ice creams, chocolate, coffee, or “culinary” products such as mayonnaise, tomato puree or salad dressings.

The composition according to the invention may also be intended for animals, especially pets, such as cats and dogs, and may be formulated in the form of feed or food supplements for animals.

According to one preferred embodiment, a probiotic microorganism that is suitable for use in the invention may be used parenterally, in particular subcutaneously or intradermally.

In such a use, a probiotic microorganism that is suitable for use in the invention may be conditioned in the form of an aqueous or non-aqueous sterile isotonic solution, in the form of a dispersion, a suspension or an emulsion prepared, where appropriate, just before administration, using a sterile powder, for example a lyophilized powder, which is then reconstituted in the form of an injectable sterile solution or a dispersion at the time of use.

The term “sterile” qualifies a formulation that is capable of ensuring the harmlessness required for intraepidermal and/or intradermal and/or subcutaneous administration.

A composition that is suitable for use in the invention may comprise any excipient usually used in the field of injectable sterile solutions.

The parenteral administration of a composition of the invention may be performed via any injection technique that is suitable for intraepidermal and/or intradermal and/or subcutaneous injection. Thus, such an administration may be performed by means of a needle usually used for performing an intraepidermal and/or intradermal and/or subcutaneous injection, suitable for mesotherapy.

Additional Active Agent

A probiotic microorganism according to the invention may advantageously be used in combination with an additional active agent, especially a cosmetic or pharmaceutical active agent.

Advantageously, such an additional cosmetic or pharmaceutical active agent may be intended for exerting a cosmetic, care or hygienic effect on the skin, the hair, the eyelashes, bodily hair and/or the scalp, and preferentially on the skin.

The additional active agents are chosen by a person skilled in the art such that they do not harm the effect of the probiotic microorganisms of the invention.

In particular, an additional active agent that is suitable for use in the invention may be chosen from active agents for reinforcing the cutaneous barrier.

According to another embodiment, active agents for preventing and/or treating skin complaints may be combined with a microorganism according to the invention.

As additional active agents that may be used, mention may be made of:
vitamins, such as vitamins A, B5, B6, B8, C, D, E or PP (vitamin B3 or niacin),
antioxidants, such as curcuminoids; carotenoids, especially a carotenoid chosen from β-carotene, lycopene, astaxanthin, zeaxanthin and lutein; polyphenol compounds, flavonoids such as catechins; proanthocyanidins, anthocyanins and PCOs (procyannidol oligomers); ubiquinones; coffee extracts containing polyphenols and/or diterpenes; chicory extracts; Ginkgo biloba extracts; proanthocyanidin-rich grape extracts; pimento extracts; soybean extracts,
minerals, such as zinc, calcium, magnesium, copper, iron, iodine, manganese, selenium or chromium (III),
sugars,
amino acids, especially sulfur-containing amino acids, such as glutathione precursors, taurine, and selenium-containing amino acids,
3 and 6 polyunsaturated fatty acids,
prebiotics, chosen especially from oligosaccharides, produced from glucose, galactose, xylose, maltose, sucrose, lactose, starch, xylan, hemicellulose, inulin, gums of acacia type, for example, or a mixture thereof. More particularly, the oligosaccharide comprises at least one fructo-oligosaccharide. More particularly, this prebiotic may comprise a mixture of fructo-oligosaccharide and inulin,
phytosterols, for instance resveratrol,
hesperidin, and
mixtures thereof.

According to one embodiment, proteins or protein hydrolysates, amino acids, polyols, especially C₂ to C₁₀ polyols, for instance glycerol, sorbitol, butylene glycol and polyethylene glycol, urea, allantoin, sugars and sugar derivatives, water-soluble vitamins, starch, and bacterial or plant extracts such as those of Aloe vera, may be used more particularly in the topical galenical forms as additional hydrophilic active agents.

According to another embodiment, retinol (vitamin A) and derivatives thereof, tocopherol (vitamin E) and derivatives thereof, ceramides, essential oils and unsaponifiable materials (tocotrienol, sesamine, gamma-oryzanol, phytosterols, squalenes, waxes and terpenes) may be used more particularly in the topical galenical forms as additional lipophilic active agents.

As additional active agents in an oral galenical form, any commonly used and/or permitted ingredient may also be considered.

By way of illustration, mention may be made of vitamins, minerals, essential fats, trace elements, polyphenols, flavonoids, phytoestrogens, antioxidants such as lipoic acid and coenzyme Q10, carotenoids, prebiotics, proteins and amino acids, monosaccharides and polysaccharides, amino-sugars, phytosterols and triterpene alcohols of plant origin.

This in particular concerns vitamins A, C, D, E and PP and B group vitamins, especially B5, B6 and B8.

Among the carotenoids, β-carotene, lycopene, lutein, zeaxanthin and astaxanthin are preferably chosen.

The minerals and trace elements particularly used are zinc, calcium, magnesium, copper, iron, iodine, manganese, selenium and chromium (III).

Among the polyphenols, polyphenols from grape, tea, olive, cocoa, coffee, apple, blueberry, elderberry, strawberry, cranberry and onion may be selected in particular.

Preferably, among the phytoestrogens, isoflavones in free or glycosylated form are selected, such as genistein, daidzein, glycitein or lignans, in particular those from flax and from Schizandra chinensis.

Preferably, among the amino acids that are suitable for use in the invention, taurine, threonine, cysteine, tryptophan or methionine, or peptides and proteins containing them, may be chosen.

Preferably, among the lipids that are suitable for use in the invention, lipids belonging to the group of oils containing monounsaturated and polyunsaturated fatty acids, such as oleic acid, linoleic acid, α-linolenic acid, γ-linolenic acid, stearidonic acid, long-chain fish omega-3 fatty acids such as EPA and DHA, conjugated fatty acids obtained from plants or animals such as CLA (Conjugated Linoleic Acid), may be chosen.

Thus, a composition intended for oral administration may also comprise at least one nutritional active agent chosen from lycopene, vitamin C, vitamin E and polyphenol compounds.

An oral composition of the invention may also comprise other nutritional active agents chosen from:
anti-ageing nutritional active agents, such as food antioxidants, nutrients with radical-scavenging properties and cofactors of antioxidant endogenous enzymes, vitamins A, C and E, carotenoids, xanthophylls, isoflavones, certain minerals such as zinc, copper, magnesium or selenium, lipoic acid, coenzyme Q10, superoxide dismutase (SOD) or taurine. Among the anti-ageing active agents, mention may be made especially of unsaponifiable fractions extracted from fats of plant origin, Aloe vera, native or hydrolysed marine collagen, and plant or marine oils rich in omega-3 and omega-6 fatty acids (including gamma-linolenic acid),
photoprotective nutritional active agents such as antioxidants and free-radical scavengers: vitamins A, C and E, carotenoids, xanthophylls, certain minerals such as zinc, copper, magnesium or selenium, coenzyme Q10 and superoxide dismutase (SOD),
nutritional ingredients with moisturizing or immunomodulatory properties such as the extract of Polypodium leucotomos, plant or marine oils rich in omega-3 or omega-6 fatty acids, including γ-linolenic acid.

According to one embodiment, the invention relates to a cosmetic process for preventing and/or treating impairment of the skin's microrelief in the case of an individual in need thereof, comprising at least one step of administering to the said individual at least one probiotic microorganism, especially of the genus Lactobacillus sp.

According to the invention, a process suitable for use in the invention may comprise a step that consists in observing a reduction in the impairment of the microrelief, or even an improvement in the skin's microrelief.

A process according to the invention may especially be performed topically by administration of a topical cosmetic and/or dermatological composition as defined above.

Advantageously, a process of the invention comprising topical application may comprise the application of a composition comprising at least one probiotic microorganism in accordance with the invention, for example in the form of a mask on the skin.

Such an administration may be performed according to the usual techniques for using these compositions. For example, it may consist in applying creams, gels, sera or lotions to the skin or mucous membranes.

A topical cosmetic process according to the invention may, for example, be performed daily, for example at a rate of one administration per day or an administration twice a day, for example once in the morning and once in the evening.

A topical cosmetic process according to the invention may be performed over a period of time ranging from one week to several weeks, or even several months, this period moreover possibly being repeated after periods without treatment, for several months or even several years.

By way of example, the topical administration of a probiotic microorganism according to the invention may be repeated, for example, 2 to 3 times a day, or more, and generally over a prolonged period of at least 4 weeks, or even 4 to 15 weeks, with, where appropriate, one or more periods of stoppage.

A cosmetic process according to the invention may be performed orally, especially by administration of a food composition as defined above.

An effective amount of microorganism may be administered in a single dose per day or in fractional doses over the day, for example two to three times a day.

An oral cosmetic process may be performed over a time period ranging from one week to several weeks, or even several months, this period moreover possibly being repeated after periods without treatment, for several months or even several years.

By way of example, the oral administration of a probiotic microorganism according to the invention may be performed at a rate, for example, of 3 times a day or more, generally over a prolonged period of at least 4 weeks, or even 4 to 15 weeks, optionally comprising one or more periods of stoppage or being repeated after a period of stoppage.

### FIGURES

FIG. 1: Illustrates the change in the number of grooves of the microrelief at the corner of the eye over time, at D1, D29 and D57 in the case of individuals of the ST11 group (light-grey line and symbols) and in the case of individuals of the placebo group (dark-grey line and symbols). The number of grooves of the microrelief in the region of the crow's feet is markedly reduced in the case of individuals of the ST11 group. The results are expressed as a mean ±confidence interval.

FIG. 2: Illustrates the change in the depth of the grooves of the microrelief in the region of the crow's feet over time, at D1, D29 and D57 in the case of individuals of the ST11 group (light-grey line and symbols) and in the case of individuals of the placebo group (dark-grey line and symbols). The depth of the grooves of the microrelief in the region of the crow's feet is markedly reduced in the case of individuals of the ST11 group. The results are expressed as a mean ±confidence interval.

FIG. 3: Change in the number of corners over time, at D1, D29 and D57 in the case of individuals of the ST11 group (light-grey line and symbols) and in the case of individuals of the placebo group (dark-grey line and symbols). The number of corners tends to increase markedly in the case of individuals of the ST11 group. The results are expressed as a mean ±confidence interval.

In the description and in the examples that follow, unless otherwise mentioned, the percentages are weight percentages and the ranges of values worded in the form “between . . . and . . . ” include the stated lower and upper limits.

The ingredients are mixed together, before being formed, in the order and under conditions that may be readily determined by a person skilled in the art.

The content and nature of the ingredients used in the compositions of the invention are adjusted by a person skilled in the art so as not to substantially affect the properties required for the compositions of the invention.

The examples below are presented as non-limiting illustrations of the invention.

### EXAMPLES

### Example 1

Effect of a Food Supplement Comprising Lactobacillus paracasei ST11 on the Skin's Microrelief

Materials and Methods

The effect of a food supplement comprising Lactobacillus paracasei ST11 on the skin's microrelief was determined by performing a double-blind randomized controlled comparative single-centre study, on two parallel groups of 33 individuals: food supplement (ST11 group) vs placebo (placebo group).

The measurements on the cutaneous microrelief were performed using a Skin Chip® machine and by echography.

The individuals selected for the study are healthy males over 60 years old, having at the start of the study (D0) a score of 4 or 5 on the Densiscore® and, in principle, being low consumers of fermented products (less than 125 g/day).

During the study, the volunteers agreed not to consume any fermented products containing live bacteria (yoghurts, cottage cheeses, fermented milks, unpasteurized cheeses, etc.).

The food supplement was formulated in the form of a powder comprising live Lactobacillus paracasei NCC 2461 (ST11) at a total dose of 10⁹ cfu and maltodextrin. The powder is to be dispersed or dissolved in a drinkable liquid, preferably water.

The placebo was formulated in the form of a powder comprising only malto-dextrin. The powder is to be dispersed or dissolved in a drinkable liquid, preferably water.

The treatment was administered once a day, for 61 days. The measurements were taken on D-42 (preinclusion), D0, D1, D2, D3, D4, D5, D29, D30, D31, D32, D33, D57, D58, D59, D60 and D61.

An analysis of the cutaneous microrelief was performed by producing silicone polymer (Silflo®) imprints of the corner of the eye, at the start of the study and at each measurement time, and measurement using a Skin Image Analyser® (SIA®) according to the following protocol.

The surface of the replica was illuminated with oblique light at 35° to generate projected shadows, which were then observed using a CCD camera connected to a computer system. The field studied was 1 cm². The digital image obtained was analysed in greyscale to obtain the various parameters characterizing the relief of the skin's surface.

The parameters studied using the Quantirides® software (Monaderm) were the total wrinkled area, and the number and average depth of the grooves of the skin's microrelief.

The maximum depth of the grooves of the microrelief was set at 55 μm. The minimum area of a groove for it to be taken into account was set arbitrarily at 0.03 mm².

An evaluation of the microrelief of the skin on the left forearm was also performed using a Skin Chip® machine.

The Skin Chip® machine is constituted by an ST Microelectronics sensor matrix (256×360 at 500 dpi) measuring the electrical permittivity. This property depends on the skin/sensor distance and on the conduction properties of the skin and thus, especially, on its moisturization. Each sensor of the matrix gives information coded in 8 bits (0 to 255), which, when converted into greyscale, constitutes an image that allows visualization of the texture and microrelief of the skin (black pixels: surface of the probe in contact with the skin; white pixels: probe remote from the skin (bottom of a groove of the microrelief)).

Results

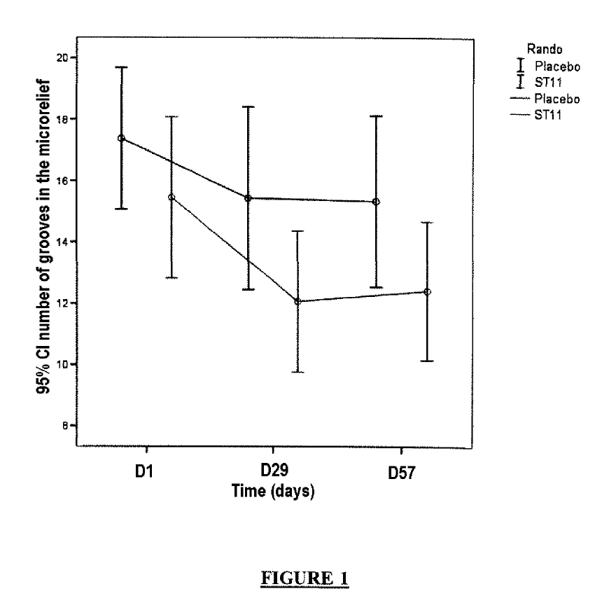

Number of Grooves in the Microrelief of the Facial Skin (FIG. 1)

Silicone polymer (Silflo®) imprints of the corner of the eye were made at the start of the study and at each measuring time, and were then studied using the Skin Image Analyser® (SIA®). The maximum depth of the grooves of the microrelief was set at 55 μm.

The measurements obtained for the ST11 and placebo groups appear comparable on D1 (p=0.2585).

Only the volunteers of the ST11 group observed a significant decrease in the number of grooves of the microrelief in the corner of the eye over time (from D29). After the supplementation, a 19.6% decrease in the number of grooves of the microrelief of the crow's feet is observed for the ST11 group, and a decrease of 11.8% for the placebo group.

The change between D1 and D57 does not appear to be different between the two treatments (p=0.5181), although a pronounced tendency in favour of the ST11 group may be observed.

TABLE 1
Test of intra-group and inter-group comparison (p-values) of the
change in the number of grooves of the microrelief in the corner
of the eye relative to D1 at all the times:

| | Placebo | | Placebo/ST11 (p |
|---|---|---|---|
| Time | (p value) | ST11 (p value) | value) |
| D1-D29 | 0.3132 | 0.0060 | 0.4022 |
| D1-D57 | 0.2080 | 0.0137 | 0.5181 |

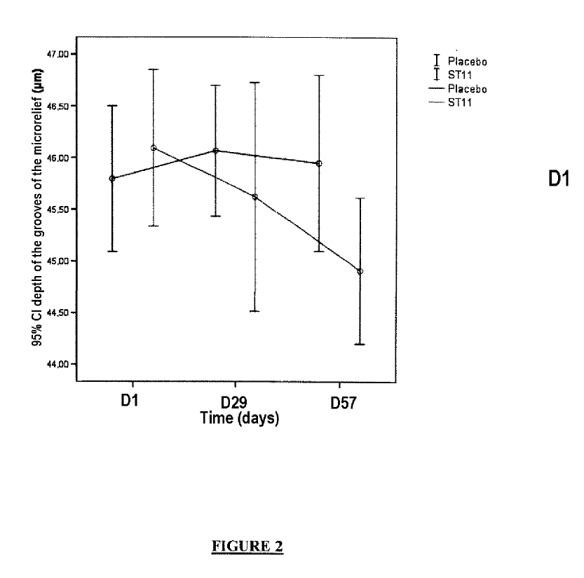

Depth of the Grooves of the Facial Microrelief (FIG. 2)

The measurements were taken on the imprints of the corner of the eye as described previously.

The measurements obtained between the ST11 group and the placebo group appear comparable at D1 (p=0.5533).

Only the volunteers of the ST11 group observed a significant decrease in the depth of the grooves of the microrelief in the corner of the eye between D1 and D57. After the supplementation, a 2.6% decrease in the depth of the grooves of the microrelief is observed for the individuals of the ST11 group, and a 0.3% decrease for the individuals of the placebo group.

The change between D1 and D57 between the two groups is different in tendency (p=0.0556).

TABLE 2
Tests of intra-group and inter-group comparison (p-values) of the
change in the depth of the grooves of the microrelief in the corner
of the eye relative to D1 at all the times:

| | | | Placebo/ST11 (p |
|---|---|---|---|
| Time | Placebo (p value) | ST11 (p value) | value) |
| D1-D29 | 0.5897 | 0.3781 | 0.3082 |
| D1-D57 | 0.7554 | 0.0185 | 0.0556 |

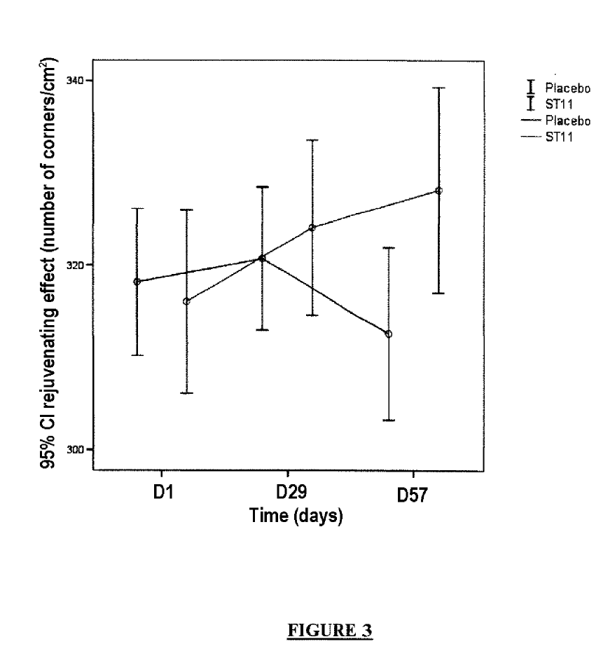

Analysis of the Microrelief on the Forearm Using a Skin-Chip® Machine (FIG. 3)

The overall microrelief is evaluated by measuring the corner density parameter or number of corners/cm². This parameter represents the number of mutual crosses of the primary fine lines per cm² and is indicative of the orientation of the fine lines of the microrelief. A high value of this parameter is characteristic of youthful, smooth, healthy skin.

The measurements obtained between the ST11 group and the placebo group appear to be comparable at D1 (p=0.7316).

The number of corners/cm², an increase in which reflects a rejuvenating effect, increases significantly over time only for the individuals of the ST11 group, while in the case of the individuals of the placebo group, no significant variation is observed.

The change between D1 and D57 between the two groups is significantly different (p=0.0117) in favour of the ST11 group. This reflects a rejuvenating effect for the individuals of the ST11 group that is statistically significant relative to the placebo group.

After the supplementation with a food supplement according to the invention, a 3.8% increase in the number of corners for the individuals of the ST11 group, and an increase of only 1.8% for the placebo group.

TABLE 3
Tests of intra-group and inter-group comparison (p-values) of the
change in the number of corners/cm² relative to D1
at all the times:

| | Placebo | | Placebo/ST11 (p |
|---|---|---|---|
| Time | (p value) | ST11 (p value) | value) |
| D1-D29 | 0.5925 | 0.0543 | 0.2340 |
| D1-D57 | 0.1324 | 0.0378 | 0.0117 |

### CONCLUSION

The results obtained in the clinical study detailed above show that the oral administration of a food supplement comprising Lactobacillus paracasei (ST11) at a dose of 10⁹ cfu/day for two months makes it possible to obtain:
on the measurement of the cutaneous microrelief by SIA®:
a decrease in the number of grooves of the facial microrelief and of their depth over time only for the ST11 group. The difference between the two treatments is significant in tendency (p=0.0556) in favour of the ST11 group when the change in depth of the grooves of the microrelief is analysed between D1 and D57.
on the measurement of the cutaneous microrelief by Skin-Chip®:
an improvement in the network of the microrelief only in the case of the individuals of the ST11 group (D1/D57, p=0.0378). The difference is statistically significant in favour of the ST11 group (p=0.0117) when the change between the two treatments is analysed between D1 and D57.

### Example 2

Topical Compositions according to the Invention

### Example 2A

Facial lotion

| | weight % |
|---|---|
| Lyophilized powder of Lactobacillus paracasei ST11 | 5.00 |
| Lyophilized powder of Lactobacillus johnsonii | 5.00 |
| Anti-inflammatory agent | 0.05 |
| Antioxidant | 0.05 |
| Isopropanol | 40.0 |
| Preserving agent | 0.30 |
| Water | qs 100 |

### Example 2B

Facial care gel

| | weight % |
|---|---|
| Powder of Lactobacillus paracasei ST11 | 5.00 |
| Hydroxypropylcellulose | 5.00 |
| (Klucel H ® sold by the company Hercules) | |
| Vitamin E | 1.00 |
| Antioxidant | 0.05 |
| Isopropanol | 40.00 |
| Preserving agent | 0.30 |
| Water | qs 100 |

### Example 2C

Facial care milk

| | weight % |
|---|---|
| Lyophilized powder of Lactobacillus paracasei ST11 | 5.00 |
| Glyceryl stearate | 1.00 |
| Cetylstearyl alcohol/cetylstearyl alcohol oxyethylenated | 3.00 |
| with 30 mol of OE (Sinnowax AO ® sold by the | |
| company Henkel) | |
| Cetyl alcohol | 1.00 |
| Dimethicone (DC 200 Fluid ® sold by the company | 1.00 |
| Dow Corning) | |
| liquid petroleum jelly | 6.00 |
| Isopropyl myristate (Estol ® IMP 1514 sold by Unichema) | 3.00 |
| Glycerol | 20.00 |
| Preserving agent | 0.30 |
| Water | qs 100 |

### Example 2D

Facial care cream

| | weight % |
|---|---|
| Arachidyl behenyl alcohol/arachidylglucoside | 3.00 |
| Isohexadecane | 7.00 |
| Powder of Lactobacillus paracasei ST11 | 5.00 |
| Glycerol | 2.00 |
| Extract of Vitreoscilla filiformis | 3.00 |
| BHT | 0.05 |
| Methyl POB | 0.10 |
| Propyl POB | 0.05 |
| Water | qs 100 |

### Example 2E

Facial care gel

| | weight % |
|---|---|
| Powder of Lactobacillus paracasei ST11 | 5.00 |
| Extract of Vitreoscilla filiformis | 3.00 |
| Antioxidant | 0.05 |
| Vitamin E | 2.50 |
| Isopropanol | 40.00 |
| Preserving agent | 0.30 |
| Water | qs 100 |

### Example 3

Oral Compositions

### Example 3A

Powder in stick form

| | |
|---|---|
| Active principle | |
| Lactobacillus paracasei ST11 | 10¹⁰ cfu |
| Excipient | |
| Xanthan gum | 0.8 mg |
| Sodium benzoate | 0.2 mg |
| Maltodextrin | qs 30 g |

One stick may be taken per day.

### Example 3B

Capsule

| | mg/capsule |
|---|---|
| Lactobacillus johnsonii | 10⁸ cfu |
| Vitamin C | 60.00 |
| Magnesium stearate | 0.02 |

One to three of these wafer capsules may be taken per day.

### Example 3C

Sugar-coated tablet

| | mg/tablet |
|---|---|
| Active materials | |
| Lactobacillus paracasei ST11 | 5 × 10⁸ cfu |
| Excipient for the core of the tablet | |
| Microcrystalline cellulose | 70.0 |
| Encompress ™ | 60.0 |
| Magnesium stearate | 3.0 |
| Anhydrous colloidal silica | 1.0 |
| Coating agent | |
| Shellac | 5.0 |
| Talc | 61.0 |
| Sucrose | 250.0 |
| Polyvidone | 6.0 |
| Titanium dioxide | 0.3 |
| Colorant | 5.0 |

This type of sugar-coated tablet may be taken 1 to 3 times per day.

### Example 3D

Sugar-coated tablet

| | mg/tablet |
|---|---|
| Active materials | |
| Lactobacillus paracasei ST11 | 10⁹ cfu |
| Lactobacillus johnsonii | 10⁹ cfu |
| Excipient for the core of the tablet | |
| Microcrystalline cellulose | 70.0 |
| Encompress ™ | 60.0 |
| Magnesium stearate | 3.0 |
| Anhydrous colloidal silica | 1.0 |
| Coating agent | |
| Shellac | 5.0 |
| Talc | 61.0 |
| Sucrose | 250.0 |
| Polyvidone | 6.0 |
| Titanium dioxide | 0.3 |
| Colorant | 5.0 |

This type of sugar-coated tablet may be taken 1 to 3 times per day.

1-12. (canceled)

13. A cosmetic process for preventing and/or treating impairment of the skin's microrelief, comprising administering at least one probiotic microorganism to an individual in need thereof, wherein the probiotic microorganism is not administered as a composition comprising Lactobacillus johnsonii and, on a weight basis:
875.42 g of 1.7% whole milk,
10 g of 1.0% skimmed milk,
30 g of Nestlé LCI yoghurt,
70 g of sugar,
3 g of stabilizer,
0.08 g of tangerine flavouring,
0.93 g of Teavigo,
0.17 g of genistein TG,
0.14 g of resveratrol,
0.24 g of 7% β-carotene CWS,
10 g of ROPUFA “30” n-3 food oil.

14. The process of claim 13, wherein the probiotic microorganism is of the Lactobacillus genus.

15. The process of claim 13, wherein the process is for reducing the number of grooves of the skin's microrelief.

16. The process of claim 13, wherein the process is for reducing the depth of the grooves of the skin's microrelief.

17. The process of claim 13, wherein the skin's microrelief is the microrelief of facial skin.

18. The process of claim 13, wherein the probiotic microorganism is orally, topically or parenterally administered.

19. The process of claim 13, wherein the probiotic microorganism is orally administered.

20. The process of claim 13, wherein the probiotic microorganism is living, semi-activated, inactivated or dead.

21. The process of claim 13, wherein the probiotic microorganism is administered as a lysate.

22. The process of claim 13, wherein the probiotic microorganism is selected from the group consisting of Lactobacillus, Bifidobacterium, Cocci, yeasts, sporulating bacteria, and mixtures thereof.

23. The process of claim 13, wherein the probiotic microorganism is a Lactobacillus paracasei.

24. The process of claim 13, wherein the probiotic microorganism is Lactobacillus paracasei CNCM I-2116 (ST11).

25. The process of claim 13, wherein the probiotic microorganism is in a composition in a proportion of from 0.0001% to 30% by weight relative to the total weight of the composition.

26. The process of claim 13, wherein the probiotic microorganism is in a composition in a proportion of from 0.001% to 20% by weight relative to the total weight of the composition.

27. The process of claim 13, wherein the probiotic microorganism is in a composition in a proportion of from 0.01% to 15% by weight relative to the total weight of the composition.

28. The process of claim 13, wherein the probiotic microorganism is in a composition in a proportion of from 0.1% to 10% by weight relative to the total weight of the composition.

29. The process of claim 13, wherein the probiotic microorganism is in a composition in a proportion of from 1% to 5% by weight relative to the total weight of the composition.

30. The process of claim 13, wherein the probiotic microorganism is orally administered as a living probiotic microorganism in a composition in a proportion of from 10³ to 10¹⁵ cfu per gram of the composition.

31. The process of claim 13, wherein the probiotic microorganism is orally administered as a living probiotic microorganism in a composition in a proportion of from 10⁵ to 10¹⁵ cfu per gram of the composition.

32. The process of claim 13, wherein the probiotic microorganism is orally administered as a living probiotic microorganism in a composition in a proportion of from 10⁷ to 10¹² cfu per gram of the composition.

33. The process of claim 13, wherein the probiotic microorganism is administered in combination with an additional active agent.
